(19) United States
(12) Patent Application Publication
(10) Pub. No.: US 2004/0137065 A1
(43) Pub. Date: Jul. 15, 2004

(54) Antibiotic polymer combination/antibiotics polymer combination

(75) Inventors: Sebastian Vogt, Erfurt (DE); Matthias Schnabelrauch, Jena (DE); Klaus-Dieter Kuhn, Marburg (DE)

Correspondence Address: Norris, McLaughlin & Marcus P.A., 30th floor, 220 East 42nd Street, New York, NY, 10017, US

(73) Assignee: Heraeus Kulzer GmbH & Co. KG, Hanau (DE)

(21) Appl. No.: 10/659,894

(22) Filed: Sep. 11, 2003

(30) Foreign Application Priority Data
Sep. 11, 2002 (DE) 102 42 476.4

(51) Int. Cl. A61K 9/14

(52) U.S. Cl. 424/486

(57) ABSTRACT

An antibiotic polymer combination/antibiotics polymer combination that ensures the continuous release of antibiotics over a period of several days under physiological conditions, and that can be used in human and veterinary medicine. Surprisingly, one or more antibiotic salts, which are sparingly soluble in water, from the groups comprising aminoglycoside antibiotics, lincosamide antibiotics, tetracycline antibiotics, glycopeptide antibiotics, quinolone antibiotics and chlorhexidine, are suspended in homogeneous polymer mixtures, which comprise one or more hydrophobic, nonionic polymers from the groups comprising poly(vinyl, chloride), post-chlorinated poly(vinyl chloride), poly(vinylidene chloride), poly(vinyl fluoride), poly(vinylidene fluoride) and copolymers comprising vinyl chloride and one or more nonionic monomers, and which comprise one or more hydrophilic polymers from the groups comprising polyethers, and this suspension forms composites that exhibit the release of an active ingredient over a period of days in an aqueous medium.

The present invention pertains to an antibiotic polymer combination/antibiotics polymer combination that ensures the continuous release of antibiotics over a period of several days under physiological conditions, and that can be used in human and veterinary medicine.

In human and veterinary medicine, use is made of medical products comprising plastics in the form of drains, catheters, cover foils, and netting materials as temporary or permanent implants for absorbing secretion, flushing, covering, and fixing. A problematic feature in this connection is that microorganisms can migrate into the organism along these plastic tubes, especially in the case of drains and catheters, and they can consequently cause local infections that are capable of spreading out farther into the organism if they are not treated. Similar problems occur when using external fixing devices. In the same way, microorganisms can hereby penetrate into the organisms along the pinned regions. Problems due to infections on the implant surfaces are also known in the case of dental implants. The necessity arises from this of having to carry out infection prophylaxis or of having to combat infection when using these implants medically. This infection suppression can basically take place systemically or locally using suitable antibiotics. The systemic use of antibiotics is associated with a series of problems. Relatively high antibiotic doses are required in order to be able to reach antimicrobially effective concentrations systemically. As a result of this, undesirable damage can occur, especially with antibiotics of the aminoglycoside type and with antibiotics of the tetracycline type, because of their nephrotoxicity or ototoxicity. Thus infection suppression via the local use of antibiotics is more practical because effective local concentrations of antibiotics can be achieved in this way while avoiding high systemic concentrations of antibiotics.

The preparation and use of antibiotic polymer composites has been the subject of intensive research studies for years, and this has led to a series of patents. Thus Shepherd and Gould disclosed the coating of catheters with hydrophilic polymethacrylates and polyacrylates into which an antibiotic had been introduced for the treatment of infections although the antibiotic was not specified in detail (T. H. Shepherd, F. E. Gould: Catheter. Mar. 3, 1971, U.S. Pat. No. 3,566,874). A prolonged release system on the basis of hydrophilic hydroxyalkyl acrylates and hydroxy methacrylates, which are polymerized to give shaped objects that are equipped with antibiotics, also stems from Shepherd and Gould and was described in the 1970's (T. H. Shepherd, F. E. Gould: Dry hydrophilic acrylate or methacrylate polymer prolonged release drug implants. Dec. 1, 1974, U.S. Pat. No. 3,857,932). Klemm described plastic particles, which were assembled from polymethacrylate and polyacrylate, for treating osteomyelitis (K. Klemm: surgical synthetic-resin material and method of treating osteomyelitis. May 3, 1975, U.S. Pat. No. 3,882,858). These plastic particles were impregnated with gentamicin or another antibiotic. An advanced proposal for the preparation of bone cement, which contains gentamicin, stems from Gross et al. (A. Gross, R. Schaefer, S. Reiss: Bone cement compositions containing gentamicin. Nov. 22, 1977, U.S. Pat. No. 4,059,684). Salts such as sodium chloride, potassium chloride, sodium bromide and potassium bromide, which are readily soluble in water, are hereby added in the form of ancillary substances to a mixture comprising powdered copolymers of methyl methacrylate and methyl acrylate, methyl methacrylate, gentamicin hydrochloride and/or gentamicin sulfate. This mixture was polymerized via peroxides. The salts that are readily soluble in water dissolve after introducing the bone cement into a physiological medium, and they leave cavities behind. Batich et al. described a new copolymer-based release system, which was synthesized with use being made of weakly acidic monomers, and which begins to swell starting from a pH value of 8.5, and which, as a result, is said to permit the release of enclosed pharmaceutially active ingredients (C. D. Batich, M. S. Cohen, K. Foster: Compositions and devices for controlled release of active ingredients. Oct. 10, 1996, U.S. Pat. No. 5,554,147).

The antimicrobial coating of medical products with antibiotic polymer systems was the subject of a series of further studies. Thus Conway et al. developed a polymer matrix comprising a silicone within which water-soluble, nitrofuran-based, active ingredients were enclosed in finely divided form (A. J. Conway, P. J. Conway, R. D. Fryar Jr.: Sustained release bactericidal cannula. Nov. 16, 1993, U.S. Pat. No. 5,261,896). The use of a matrix-forming polymer from the group comprising polyurethanes, silicones and biodegradable polymers, in which a mixture comprising a silver salt and chlorhexidine is suspended, was disclosed for the preparation of infection-resistant medical products (C. L. Fox Jr., S. M. Modak, L. A. Sampath: Infection-resistant compositions, medical devices and surfaces and methods for preparing and using same. May 28, 1991, U.S. Pat. No. 5,019,096). Similar anti-infective systems on the basis of polyurethane and chlorhexidine dispersed therein were proposed by Solomon, Byron and Parke (D. D. Solomon, M. P. Byron: Anti-infective and antithrombogenic medical articles and method for their preparation. Sep. 19, 1995, U.S. Pat. No. 5,451,424; D. D. Solomon, M. P. Parke: Anti-infective and antithrombogenic medical articles and method for their preparation. Jan, 13, 1998, U.S. Pat. No. 5,707,366; D. D. Solomon, M. P. Parke: Anti-infective and antithrombogenic medical articles and method for their preparation. Jan. 13, 1998, U.S. Pat. No. 5,165,952). It was possible to process these systems to give shaped objects via extrusion from the melt. An antibiotic composition, which is composed of oligodynamically active metals and polymers, has also been disclosed (D. Laurin, J. Stupar: Antimicrobial compositions. Jul. 29, 1984, U.S. Pat. No. 4,603,152). Acrylonitrile/butadiene/styrene copolymers, poly(viyl chloride), polyesters, polyurethanes, styrene block copolymers and rubber are proposed as the polymers into which oligodynamically active metals are introduced in order to suppress infection. Elastomers can also be antibiotically equipped. Thus Allen produced elastomer/active ingredient combinations by blending together the active ingredients and incorporating them into master batches of rubber (D. L. Allen: Elastomeric composition containing therapeutic agents and articles manufactured therefrom. Jun. 28, 1991, U.S. Pat. No. 5,019,378). The master batches are composed of rubber, mica, and titanium dioxide. An antibiotic coating comprising a mixture of rifampin and minocycline, which had been dispersed in a polymer, was proposed by Raad and Darouiche (I. I. Raad, R. O. Darouiche: Antibacterial coated medical implants. Jun. 8, 1993, U.S. Pat. No. 5,217,493). The polymer material is not characterized in greater detail in this regard. De Leon et al. disclose a method for the antibiotic coating of implants, whereby the surface that is to be coated is first coated with silicone oil (J. De Leon, T. H. Ferguson, D. S. Skinner Jr.: Method of making antimicrobial coated implants. Mar. 28, 1990, U.S. Pat. No. 4,952,419). The powdered active ingredient is applied to the silicone oil layer in a second step. Oxytetracycline was used as the active ingredient in this case. A similar coating on the basis of silicone oil and poly(methacrylic acid esters) was described by Takigawa, whereby this coating was prepared from a solution of silicone oil and poly(methacrylic acid esters) in turpentine oil, N-decanes, tetrachloromethane, butan-2-one, 1,4-dioxane, ethoxyethanol, and toluene (B. Takigawa: Coating solution containing silicone oil and polymethacrylate. Feb, 4, 1998, U.S. Pat. No. 5,721,301. Mustacich et al. describe an antimicrobial polymer combination, whereby fatty acids and fatty acid salts are introduced as biocidal reagents into medically usable polymers (R. V. Mustacich, D. S. Lucas, R. L. Stone: Antimicrobial polymer compositions. Oct. 30, 1984, U.S. Pat. No. 4,479,795).

An interesting coating composition has been disclosed by Whitbourne and Mangan in which, as the antimicrobial reagent, quaternary ammonium compounds are incorporated into a water-insoluble polymer, e.g. cellulose esters (R. J. Whitbourne, M. A. Mangan: Coating compositions comprising pharmaceutical agents. Jun. 11, 1996, U.S. Pat. No. 5,525,348). A series of patents by Friedmann et al. have become known that are concerned with the preparation of dental lacquers (M. Friedmann, D. Steinerg, A. Soskolne: Sustained-release pharmaceutical compositions. Jun. 11, 1991, U.S. Pat. No. 5,023,082; M. Friedman, A. Sinov: Liquid polymer composition, and method of use. Nov. 3, 1992, U.S. Pat. No. 5,160,737; M. Friedman, A. Sinov: Dental varnish composition, and method of use. Jul. 19, 1994, U.S. Pat. No. 5,330,746; M. Friedman, A. Sinov: Dental varnish composition, and method of use. Jul. 15, 1997, U.S. Pat. No. 5,648,399; M. Friedman, A. Sinov: Dental varnish composition, and method of use. Jun. 17, 1997, U.S. Pat. No. 5,639,795). These patents are virtually identical in terms of content, and they contain quaternary ammonium salts as the essential antimicrobial substances. Lacquers and polymer solutions for the preparation thereof are described in the patents, whereby these essentially comprise the following components: a copolymer which is assembled from methacrylic acid and methacrylic acid esters and which has free carboxylic acid groups; a copolymer which is assembled from methacrylic acid and methyl methacrylate and which has free carboxylic acid groups; a copolymer which is assembled from dimethylaminoethyl acrylate and ethyl methacrylate; and a copolymer which is formed from methyl acrylate and chlorotrimethylammoniumethyl methacrylate. In the case of U.S. Pat. No. 5,648,399, it is interesting that an addition is made to the polymer combination of a reagent, which influences the release of the active ingredient, from the group that comprises crosslinking reagents, polysaccharides, lipids, polyhydroxy compounds, poly(carboxylic acids), divalent cations, citric acid, sodium citrate, sodium docusate, proteins, polyoxyethylene sorbitan mono-oleate, and amino acids.

An interesting proposal for the preparation of antimicrobial medical products stems from Bayston and Grove (R. Bayston, N. J. Grove: Antimicrobial device and method. Apr. 17, 1990, U.S. Pat. No. 4,917,686). Antibiotic substances are hereby dissolved in a suitable organic solvent. This solution is allowed to act on the polymer surfaces that are to be modified. The polymer begins to swell because of the solvent, and the active ingredient can penetrate into the surface. Darouiche and Raad propose a method, which is basically the same, for the antimicrobial impregnation of catheters and other medical implants, whereby an antimicrobially active ingredient is also dissolved in an organic solvent (R. Darouche, I. Raad: Antimicrobial impregnated catheters and other medical implants and method for impregnating catheters and other medical implants with an antimicrobial agent. Apr. 29, 1997, U.S. Pat. No. 5,624,704). This solution is allowed to act on the surface that is to be treated, whereby the active ingredient penetrates into the material, and is deposited there.

An alternative to the previously described systems is represented by a method, which was described by Lee, for coating surfaces with cationic antibiotics (C. C. Lee: Coating medical devices with cationic antibiotics. Jan. 23, 1990, U.S. Pat. No. 4,895,566). In the case of this method, a negatively charged heparin layer is first applied to the surface that is to be coated, and then, after its adherence thereto, cationic antibiotics are allowed to deposit thereon. A similar solution was proposed by Greco et al. in which a solution of anionic surface active substances is first allowed to act on the surface that is to be coated (R. S. Greco, R. A. Harvey, S. Z. Trooskin: Drug bonded prosthesis and process for producing same. Nov. 7, 1989, U.S. Pat. No. 4,879,135). The anionic molecules hereby adsorb to the surface. Cationic active ingredients, such as e.g. gentamicin, are then electrostatically bound thereto. In the case of the two latter processes that were quoted, the comment should be made that the loading density with respect to antibiotics per unit surface area is very limited, and the strength of adhesion of these coatings is to be regarded as critical.

The problem that forms the basis of the present invention is to develop a flexible antibiotic polymer combination/antibiotics polymer combination that permits the continuous release of antibiotics over a period of several days to weeks under physiological conditions, and that can be used in human and veterinary medicine. This antibiotic polymer combination/antibiotics polymer combination is to be capable of being applied in a simple way and in a strongly adherent manner to the surfaces of plastic medical implants and metallic medical implants. In this regard, it is especially important that the coating is flexible and elastic, and that no toxic components are released. Moreover, the flexible antibiotic polymer combination/antibiotics polymer combination should be suitable for preparing antibiotic filaments, foils and shaped objects.

The problem is solved as described hereinbelow.

The surprising finding that forms the basis of the invention is that one or more antibiotic salts, which are sparingly soluble in water, from the groups comprising aminoglycoside antibiotics, lincosamide antibiotics, tetracycline antibiotics, glycopeptide antibiotics, quinolone antibiotics and chlorhexidine, are suspended in homogeneous polymer mixtures, which comprise one or more hydrophobic, nonionic polymers from the groups comprising poly(vinyl chloride), post-chlorinated poly(vinyl chloride), poly(vinylidene chloride), poly(vinyl fluoride), poly(vinylidene fluoride) and copolymers comprising vinyl chloride and one or more nonionic monomers, and which comprise one or more hydrophilic polymers from the groups comprising polyethers, and this suspension forms composites that exhibit the release of an active ingredient over a period of days in an aqueous medium.

Thus the problem for the invention is solved by the feature that one or more antibiotic salts, which are sparingly soluble in water, from the groups comprising aminoglycoside antibiotics, lincosamide antibiotics, tetracycline antibiotics, glycopeptide antibiotics, quinolone antibiotics and chlorhexidine, and optionally an antibiotic, which is readily soluble in water, from the groups comprising aminoglycoside antibiotics, lincosamide antibiotics, β-lactam antibiotics and tetracycline antibiotics, and optionally one or more organic ancillary substances are suspended in a homogenous polymer mixture, which comprises one or more hydrophobic, nonionic polymers from the groups comprising poly(vinyl chloride), post-chlorinated poly(vinyl chloride), poly(vinylidene chloride), poly(vinyl fluoride), poly(vinylidene fluoride) and copolymers comprising vinyl chloride and one or more nonionic monomers, and which comprises one or more hydrophilic polymers from the groups comprising polyethers, and this suspension forms a composite.

It is therefore in accordance with the invention that one or more representatives of the antibiotic salts that are sparingly soluble in water, namely gentamicin dodecyl sulfate, gentamicin dodecylsulfonate, gentamicin laurate, gentamicin decyl sulfate, amikacin dodecyl sulfate, amikacin dodecylsulfonate, amikacin laurate, kanamycin dodecyl sulfate, kanamycin dodecylsulfonate, kanamycin laurate, kanamycin myristate, tobramycin dodecyl sulfate, tobramycin dodecylsulfonate, tobramycin laurate, tobramycin myristate, vancomycin dodecyl sulfate, vancomycin laurate, vancomycin myristate, teicoplanin/vancomycin, clindamycin laurate, tetracycline dodecyl sulfate, tetracycline laurate, minocycline dodecyl sulfate, minocycline laurate, oxytetracycline dodecyl sulfate, oxytetracycline laurate, rolitetracycline laurate, rolitetracycline dodecyl sulfate, chlortetracycline dodecyl sulfate, chlortetracycline laurate, ciprofloxacin laurate, ciprofloxacin myristate, moxifloxacin myristate, chlorhexidine dodecyl sulfate, chlorhexidine laurate and chlorhexidine caprate, and optionally an antibiotic, which is readily soluble in water, from the groups comprising aminoglycoside antibiotics, lincosamide antibiotics, β-lactam antibiotics and tetracycline antibiotics, and optionally one or more organic ancillary substances are suspended in a homogenous polymer mixture, which comprises one or more hydrophobic, nonionic polymers from the groups comprising poly(vinyl chloride), post-chlorinated poly(vinyl chloride), poly(vinylidene chloride), poly(vinyl fluoride), poly(vinylidene fluoride) and copolymers comprising vinyl chloride and one or more nonionic monomers, and which comprises one or more hydrophilic polymers from the groups comprising polyethers, and this suspension forms a composite. The use of other antibiotics and antimicrobial chemotherapeutic agents, which are sparingly soluble in an aqueous medium, also forms part of the basic idea of the invention.

It is advantageous if the composite comprises a free-flowing suspension, which comprises a homogeneous mixture of cyclohexanone and/or tetrahydrofuran and optionally plasticizers from the groups comprising the esters of phthalic acid, the esters of trimellitic acid, the esters of phosphoric acid, the esters of adipic acid, the esters of azelaic acid, the esters of sebacic acid, and one or more hydrophobic, nonionic polymers from the groups comprising poly(vinyl chloride) and copolymers comprising vinyl chloride and one or more nonionic monomers, and one or more hydrophilic polymers from the groups comprising polyethers, whereby, as a result of the evaporation of the cyclohexanone and/or tetrahydrofuran, the following are suspended in this free-flowing suspension: one or more antibiotic salts, which are sparingly soluble in water, from the groups comprising aminoglycoside antibiotics, lincosamide antibiotics, tetracycline antibiotics, quinolone antibiotics and chlorhexidine, and optionally an antibiotic, which is readily soluble in water, from the groups comprising aminoglycoside antibiotics, lincosamide antibiotics, β-lactam antibiotics and tetracycline antibiotics, and optionally one or more organic ancillary substances.

It is also advantageous if use is made of mixtures of cyclohexanone and tetrahydrofuran, and if other organic solvents and solvent mixtures are used that are capable of dissolving poly(vinyl chloride).

It is also advantageous if the composite is formed from a melt that comprises one or more hydrophobic, nonionic polymers from the groups comprising poly(vinyl chloride) and/or copolymers comprising vinyl chloride and one or more nonionic monomers, and one or more hydrophilic polymers from the groups comprising polyethers, and optionally plasticizers from the groups comprising the esters of phthalic acid, the esters of trimellitic acid, the esters of phosphoric acid, the esters of citric acid, the esters of tartaric acid, the esters of malic acid, the esters of fatty acids, the esters of adipic acid, the esters of azelaic acid, the esters of sebacic acid, whereby the following are suspended in this melt: one or more antibiotic salts, which are sparingly soluble in water, from the groups comprising aminoglycoside antibiotics, lincosamide antibiotics, tetracycline antibiotics, quinolone antibiotics and chlorhexidine, and optionally an antibiotic, which is readily soluble in water, from the groups comprising aminoglycoside antibiotics, lincosamide antibiotics, and tetracycline antibiotics, and optionally one or more organic ancillary substances.

It is also expedient if the quantity of hydrophilic polymer in the homogeneous polymer mixture amounts to between 0.1 and 60 percent by weight.

It is especially advantageous if poly(ethylene glycol) with a number average molecular weight in the range from 120 gmol⁻¹ to 35,000 gmol⁻¹ is used as the polyether.

It likewise advantageous if poly(propylene glycol) with a number average molecular weight in the range from 200 gmol⁻¹ to 35,000 gmol⁻¹ is used as the polyether.

Poly(ethylene glycol) with a number average molecular weight in the range from 200 gmol⁻¹ to 600 gmol⁻¹ is expediently used as the polyether.

It is likewise advantageous if vinyl chloride copolymers with number average molecular weightes from 20,000 gmol⁻¹ to 2,000,000 gmol⁻¹ are used as the hydrophobic polymers, whereby these vinyl chloride copolymers are prepared from vinyl chloride and the following comonomers: vinylidene chloride, vinyl fluoride, vinyl acetate, acrylonitrile, aliphatic esters of acrylic acid, aromatic esters of acrylic acid, aliphatic esters of methacrylic acid, aromatic esters of methacrylic acid, ethene, propene, butadiene, isoprene, 2-chlorobutadiene, and isopropylene.

It is also meaningful if sulfonamides and/or antiphlogistic substances and/or anesthetic substances and/or vancomycin hydrochloride are preferred as organic ancillary substances.

It is also expedient if the free-flowing suspension forms composites in the form of filaments as a result of spinning together with the evaporation of the cyclohexanone and/or tetrahydrofuran, or that the free-flowing suspension forms composites in the form of foils as a result of casting together with the evaporation of the cyclohexanone and/or tetrahydrofuran, or that the free-flowing suspension forms composites in the form of powders and granulated materials as a result of spraying together with the evaporation of the cyclohexanone and/or tetrahydrofuran.

It is advantageous in regard to the basic idea of the invention if the composite is formed by compressing, extruding, and rolling to give shaped objects and foils.

It is expedient if the plastic tubes, plastic filaments, plastic foils, spherical plastic objects, roller-like plastic objects, and chain-like plastic objects, which are coated with the composite, are used as medical implants.

It also is expedient if the catheters, tracheal cannulas, and tubes for intraperitoneal feeding are coated with the composite, or if implantable metal plates, metal nails, and metal screws are coated with the composite.

A feature that also forms part of the basic idea of the invention is that the composite is used for gluing medically usable shaped plastic objects, plastic foils, plastic filaments, metal plates, and metal pipes.

It is advantageous if the composite is used as a binder for preparing antibiotic shaped objects comprising granulated plastic materials, plastic powders, resorbable glass powders, non-resorbable glass powders, and quartz powders.

It is also advantageous if the free-flowing suspension is applied to the surface of plastics and/or metals via immersion, spraying, painting, brushing or rolling, and a composite in the form of a coating is formed via the evaporation of the cyclohexanone and/or tetrahydrofuran.

It is also meaningful if the composite is applied in the form of a coating to medically usable plastic filaments, plastic foils, plastic tubes, plastic pouchcs, and plastic bottles It is preferred in accordance with the invention if the composite is applied in the form of a coating to spherical shaped objects, to roller-like shaped objects, and to chain-like shaped objects, whereby these comprise plastic and/or metal.

It is also expedient if the composite is applied in the form of a coating to shaped objects, foils, and filaments comprising poly(methacrylic acid esters), poly(acrylic acid esters) poly(methacrylic acid esters-co-acrylic acid esters), poly(vinyl chloride), poly(vinylidene chloride), silicone, polystyrene, and polycarbonate.

It is likewise expedient if the composite is used as a binder for the preparation of antibiotic laminates.

It is also advantageous if the composite is applied in the form of a coating to the surface of metals and/or plastics via sintering.

The invention will be elucidated in more detail by means of two examples.

### EXAMPLE

A solution is prepared that comprises 1.50 g of poly(vinyl chloride), 300 mg of poly(ethylene glycol) 600, and 13.50 g of cyclohexanone. 1.00 g of gentamicin sulfate (AK 628) is then dissolved separately in 1 mL of distilled water. 0.50 g of sodium dodecyl sulfate is then dissolved separately in 0.75 mL of water. The aqueous solution of gentamicin sulfate is first added, drop by drop, to the poly(vinyl chloride)/PEG600/cyclohexanone solution with stirring, followed by the aqueous solution of sodium dodecyl sulfate. A 2.5 cm long piece of conventional PVC Redon tube (d=6 mm) is immersed in the suspension that is produced, and then dried at room temperature, whereby the cyclohexanone evaporates. A strongly adhering coating is produced on the surface of the tube. The mass of the coating amounts to 16 mg.

### EXAMPLE 2

A solution is prepared that comprises 1.50 g of poly(vinyl chloride), 300 mg of poly(ethylene glycol) 600, and 13.50 g of cyclohexanone. 0.59 g of gentamicin sulfate (AK 628) is then dissolved in 1 mL of distilled water. 0.25 g of sodium dodecyl sulfate is then dissolved in 0.75 mL of water. The aqueous solution of gentamicin sulfate is first added, drop by drop, to the poly(vinyl chloride)/PEG600/cyclohexanone solution with stirring, followed by the aqueous solution of sodium dodecyl sulfate. A 2.5 cm long piece of conventional PVC Redon tube (d=6 mm) is then sprayed with the suspension that had been produced using a conventional spray pistol with compressed air. The sprayed PVC tube is allowed to dry at room temperature. After the cyclohexanone has evaporated, a coating is produced that strongly adheres to the surface of the tube. The mass of the coating amounts to 18 mg.

Gentamicin Release Experiments

The pieces of tube that were coated in Examples 1 and 2 were introduced into a pH 7.4 Sorensen buffer, and this was stored over a period of four weeks at 37° C. in order to determine the retarded release of the antibiotics. The removal of samples took place after 1, 2, 3, 4, 5, and 6 days of storage. The determination of the level of the antibiotics was carried out using a microbial agar diffusion test with use being made of bacillus subtilis ATCC 6633 as the test germ.

TABLE 1
Cumulative release of gentamicin sulfate from the coated
tubes from Examples 1 and 2 as a function of the time of
storage in physiological sodium chloride solution at 37° C.

Cumulative release of gentamicin sulfate
[μg] (as gentamicin sulfate AK628)
Storage time [d]

| Examples | 1 | 2 | 3 | 4 | 5 | 6 |
|---|---|---|---|---|---|---|
| 1 | 1830 | 2170 | 2210 | 2230 | 2250 | 2260 |
| 2 | 1360 | 1440 | 1450 | 1460 | 1480 | 1490 |

1. Antibiotic polymer combination/antibiotics polymer combination, comprising (a) one or more antibiotic salts, which are sparingly soluble in water, from the groups comprising aminoglycoside antibiotics, lincosamide antibiotics, tetracycline antibiotics, glycopeptide antibiotics, quinolone antibiotics and chlorhexidine, and optionally (b) an antibiotic, which is readily soluble in water, from the groups comprising aminoglycoside antibiotics, lincosamide antibiotics, β-lactam antibiotics and tetracycline antibiotics, and optionally (c) one or more organic ancillary substances suspended in a homogenous polymer mixture to form a suspension wherein the homogeneous polymer mixture comprises one or more hydrophobic, nonionic polymers from the groups comprising poly(vinyl chloride), post-chlorinated poly(vinyl chloride), poly(vinylidene chloride), poly(vinyl fluoride), poly(vinylidene fluoride) and copolymers comprising vinyl chloride and one or more nonionic monomers, and which comprises one or more hydrophilic polymers from the groups comprising polyethers, and wherein the suspension forms a composite.

2. Antibiotic polymer combination/antibiotics polymer combination, (a) one or more representatives of the antibiotic salts that are sparingly soluble in water, namely gentamicin dodecyl sulfate, gentamicin dodecylsulfonate, gentamicin laurate, gentamicin decyl sulfate, amikacin dodecyl sulfate, amikacin dodecylsulfonate, amikacin laurate, kanamycin dodecyl sulfate, kanamycin dodecylsulfonate, kanamycin laurate, kanamycin myristate, tobramycin dodecyl sulfate, tobramycin dodecylsulfonate, tobramycin laurate, tobramycin myristate, vancomycin dodecyl sulfate, vancomycin laurate, vancomycin myristate, teicoplanin/vancomycin, clindamycin laurate, tetracycline dodecyl sulfate, tetracycline laurate, minocycline dodecyl sulfate, minocycline laurate, oxytetracycline dodecyl sulfate, oxytetracycline laurate, rolitetracycline laurate, rolitetracycline dodecyl sulfate, chlortetracycline dodecyl sulfate, chlortetracycline laurate, ciprofloxacin laurate, ciprofloxacin myristate, moxifloxacin myristate, chlorhexidine dodecyl sulfate, chlorhexidine laurate and chlorhexidine caprate, and optionally (b) an antibiotic, which is readily soluble in water, from the groups comprising aminoglycoside antibiotics, lincosamide antibiotics, P-lactam antibiotics and tetracycline antibiotics, and optionally (c) one or more organic ancillary substances suspended in a homogenous polymer mixture to form a suspension wherein the homogenous polymer mixture comprises one or more hydrophobic, nonionic polymers from the groups comprising poly(vinyl chloride), post-chlorinated poly(vinyl chloride), poly(vinylidene chloride), poly(vinyl fluoride), poly(vinylidene fluoride) and copolymers comprising vinyl chloride and one or more nonionic monomers, and which comprises one or more hydrophilic polymers from the groups comprising polyethers, and wherein the suspension forms a composite.

3. Antibiotic polymer combination/antibiotics polymer combination in accordance with claim 1 wherein the composite is formed from a free-flowing suspension, which comprises a homogeneous mixture of cyclohexanone and/or tetrahydrofuran and optionally plasticizers from the groups comprising the esters of phthalic acid, the esters of trimellitic acid, the esters of phosphoric acid, the esters of adipic acid, the esters of azelaic acid, the esters of sebacic acid, and one or more hydrophobic, nonionic polymers from the groups comprising poly(vinyl chloride) and copolymers comprising vinyl chloride and one or more nonionic monomers, and one or more hydrophilic polymers from the groups comprising polyethers, whereby, as a result of evaporation of the cyclohexanone and/or tetrahydrofuran, the following are suspended in this free-flowing suspension: one or more antibiotic salts, which are sparingly soluble in water, from the groups comprising aminoglycoside antibiotics, lincosamide antibiotics, tetracycline antibiotics, quinolone antibiotics and chlorhexidine, and optionally an antibiotic, which is readily soluble in water, from the groups comprising aminoglycoside antibiotics, lincosamide antibiotics, β-lactam antibiotics and tetracycline antibiotics, and optionally one or more organic ancillary substances.

4. Antibiotic polymer combination/antibiotics polymer combination in accordance with claim 1, wherein the composite is formed from a melt that comprises one or more hydrophobic, nonionic polymers from the groups comprising poly(vinyl chloride) and/or copolymers, which comprise vinyl chloride and one or more nonionic monomers, and one or more hydrophilic polymers from the groups comprising polyethers, and optionally plasticizers from the groups comprising the esters of phthalic acid, the esters of trimellitic acid, the esters of phosphoric acid, the esters of citric acid, the esters of tartaric acid, the esters of malic acid, the esters of fatty acids, the esters of adipic acid, the esters of azelaic acid, the esters of sebacic acid, whereby the following are suspended in this melt: one or more antibiotic salts, which are sparingly soluble in water, from the groups comprising aminoglycoside antibiotics, lincosamide antibiotics, tetracycline antibiotics, quinolone antibiotics and chlorhexidine, and optionally an antibiotic, which is readily soluble in water, from the groups comprising aminoglycoside antibiotics, lincosamide antibiotics, and tetracycline antibiotics, and optionally one or more organic ancillary substances.

5. Antibiotic polymer combination/antibiotics polymer combination in accordance with claim 1, wherein the quantity of hydrophilic polymer in the homogeneous polymer mixture amounts to between 0.1 and 60 percent by weight.

6. Antibiotic polymer combination/antibiotics polymer combination in accordance with claim 1, wherein poly(ethylene glycol) with a number average molecular weight in the range from 120 gmol⁻¹ to 35,000 gmol⁻¹ is used as the polyether.

7. Antibiotic polymer combination/antibiotics polymer combination in accordance with claim 1, wherein poly(propylene glycol) with a number average molecular weight in the range from 200 gmol⁻¹ to 35,000 gmol⁻¹ is used as the polyether.

8. Antibiotic polymer combination/antibiotics polymer combination in accordance with claim 1, wherein poly(ethylene glycol) with a number average molecular weight in the range from 120 gmol⁻¹ to 600 gmol⁻¹ is used as the polyether.

9. Antibiotic polymer combination/antibiotics polymer combination in accordance with claim 1, wherein vinyl chloride copolymers with number average molecular weights from 20,000 gmol⁻¹ to 2,000,000 gmol⁻¹ are used as the hydrophobic polymers, whereby these vinyl chloride copolymers are prepared from vinyl chloride and the following comonomers: vinylidene chloride, vinyl fluoride, vinyl acetate, acrylonitrile, aliphatic esters of acrylic acid, aromatic esters of acrylic acid, aliphatic esters of methacrylic acid, aromatic esters of methacrylic acid, ethene, propene, butadiene, isoprene, 2-chlorobutadiene and isopropylene.

10. Antibiotic polymer combination/antibiotics polymer combination in accordance with claim 1, wherein sulfonamides and/or antiphlogistic substances and/or anesthetic substances are used as the organic ancillary substances.

11. Antibiotic polymer combination/antibiotics polymer combination in accordance with claim 3, wherein the free-flowing suspension forms composites in the form of filaments as a result of spinning together with the evaporation of the cyclohexanone and/or tetrahydrofuran.

12. Antibiotic polymer combination/antibiotics polymer combination in accordance with claim 3, wherein the free-flowing suspension forms composites in the form of foils as a result of casting together with the evaporation of the cyclohexanone and/or tetrahydrofuran.

13. Antibiotic polymer combination/antibiotics polymer combination in accordance with claim 3, wherein the free-flowing suspension forms composites in the form of powders and granulated materials as a result of spraying together with the evaporation of the cyclohexanone and/or tetrahydrofuran.

14. Antibiotic polymer combination/antibiotics polymer combination in accordance with claim 1, wherein the composite is formed by compressing, extruding, and rolling to give shaped objects, coatings, and foils.

15. Antibiotic polymer combination/antibiotics polymer combination in accordance with claim 1, which comprises plastic tubes, plastic filaments, plastic foils, spherical plastic objects, roller-like plastic objects, or chain-like plastic objects coated with the composite.

16. Antibiotic polymer combination/antibiotics polymer combination in accordance with claim 1, which comprises catheters, tracheal cannulas, or tubes for intraperitoneal feeding coated with the composite.

17. Antibiotic polymer combination/antibiotics polymer combination in accordance with claim 1, which comprises implantable metal plates, metal nails, or metal screws coated with the composite.

18. Antibiotic polymer combination/antibiotics polymer combination in accordance with claim 1, which comprises medically usable shaped plastic objects, plastic foils, plastic filaments, metal plates, or metal pipes glued together or to a substrate with the composite.

19. Antibiotic polymer combination/antibiotics polymer combination in accordance with claim 1, which comprises antibiotic shaped objects comprising granulated plastic materials, plastic powders, resorbable glass powders, non-resorbable glass powders, or quartz powders binded with the composite.

20. Antibiotic polymer combination/antibiotics polymer combination in accordance with claim 1, which comprises antibiotic laminates binded with the composite.

21. Method of using an antibiotic polymer combination/antibiotics polymer combination in accordance with claim 3, comprising applying the free-flowing suspension to the surface of plastics and/or metals via immersion, spraying, painting, brushing or rolling, and forming a composite in the form of a coating via the evaporation of the cyclohexanone.

22. Method of using an antibiotic polymer combination/antibiotics polymer combination in accordance with claim 1, comprising applying the composite in the form of a coating to medically usable plastic filaments, plastic foils, plastic tubes, plastic pouches, or plastic bottles.

23. Method of using an antibiotic polymer combination/antibiotics polymer combination in accordance with claim 1, comprising applying the composite in the form of a coating to spherical shaped objects, to roller-like shaped objects, or to chain-like shaped objects, whereby these comprise plastic and/or metal.

24. Method of using an antibiotic polymer combination/antibiotics polymer combination in accordance with claim 1, comprising applying the composite in the form of a coating to shaped objects, foils, or filaments comprising poly(methacrylic acid esters), poly(acrylic acid esters) poly(methacrylic acid esters-co-acrylic acid esters), poly(vinyl chloride), poly(vinylidene chloride), silicone, polystyrene, or polycarbonate.

25. Method of using an antibiotic polymer combination/antibiotics polymer combination in accordance with claim 1, comprising applying the composite in the form of a coating to the surface of metals and/or plastics-via sintering.

26. Antibiotic polymer combination/antibiotics polymer combination in accordance with claim 2, wherein the composite is formed from a free-flowing suspension, which comprises a homogeneous mixture of cyclohexanone and/or tetrahydrofuran and optionally plasticizers from the groups comprising the esters of phthalic acid, the esters of trimellitic acid, the esters of phosphoric acid, the esters of adipic acid, the esters of azelaic acid, the esters of sebacic acid, and one or more hydrophobic, nonionic polymers from the groups comprising poly(vinyl chloride) and copolymers comprising vinyl chloride and one or more nonionic monomers, and one or more hydrophilic polymers from the groups comprising polyethers, whereby, as a result of evaporation of the cyclohexanone and/or tetrahydrofuran, the following are suspended in this free-flowing suspension: one or more antibiotic salts, which are sparingly soluble in water, from the groups comprising aminoglycoside antibiotics, lincosamide antibiotics, tetracycline antibiotics, quinolone antibiotics and chlorhexidine, and optionally an antibiotic, which is readily soluble in water, from the groups comprising aminoglycoside antibiotics, lincosamide antibiotics, β-lactam antibiotics and tetracycline antibiotics, and optionally one or more organic ancillary substances.

27. Antibiotic polymer combination/antibiotics polymer combination in accordance with claim 2, wherein the composite is formed from a melt that comprises one or more hydrophobic, nonionic polymers from the groups comprising poly(vinyl chloride) and/or copolymers, which comprise vinyl chloride and one or more nonionic monomers, and one or more hydrophilic polymers from the groups comprising polyethers, and optionally plasticizers from the groups comprising the esters of phthalic acid, the esters of trimellitic acid, the esters of phosphoric acid, the esters of citric acid, the esters of tartaric acid, the esters of malic acid, the esters of fatty acids, the esters of adipic acid, the esters of azelaic acid, the esters of sebacic acid, whereby the following are suspended in this melt: one or more antibiotic salts, which are sparingly soluble in water, from the groups comprising aminoglycoside antibiotics, lincosamide antibiotics, tetracycline antibiotics, quinolone antibiotics and chlorhexidine, and optionally an antibiotic, which is readily soluble in water, from the groups comprising aminoglycoside antibiotics, lincosamide antibiotics, and tetracycline antibiotics, and optionally one or more organic ancillary substances.
